(19) United States
(12) Patent Application Publication
(10) Pub. No.: US 2021/0282939 A1
(43) Pub. Date: Sep. 16, 2021

(54) PROSTHESIS EXTRACTION APPARATUS

(71) Applicant: Mark B. Wright, Rupert, ID (US)

(72) Inventor: Mark B. Wright, Rupert, ID (US)

(21) Appl. No.: 17/198,396

(22) Filed: Mar. 11, 2021

Related U.S. Application Data
(60) Provisional application No. 62/988,444, filed on Mar. 12, 2020.

(51) Int. Cl. A61F 2/46

(52) U.S. Cl. CPC A61F 2/4607; A61F 2/461; A61F 2002/4625; A61F 2002/4619; A61F 2/4612

(57) ABSTRACT

The present application is directed to a Prosthesis Extraction Apparatus having a surgical blade mounting and support block configured to be attachable to a surgical osteotome or manual handle. Surgical knife blades are secured to the blade support block and can then be removed, replaced or moved back and forth to adjust to the width of the prosthesis to be extracted by rotating the calibrated indexing wheel at the end of the support block member. Surgical knife blades will initially be used to properly align the support apparatus with the side surfaces of the existing prosthesis to be extracted. Securing of the support apparatus down against the femur end is accomplished by an alignment blade configured with a screw or barbed stem attachment threaded into the threaded orifice of the existing prosthesis to be extracted. An alignment bar may be used to provide optimal alignment of the surgical knife blades.

### FIELD OF THE INVENTION

This application relates to a unique design of new Prosthesis Extraction Apparatus to be used on joint replacement in the area of the hip, shoulder and knee. More particularly, the Prosthesis Extraction Apparatus of the present invention will be provided with the two knife blades moveable outwardly from the side of a support member, wherein the knife blades used on the Prosthesis Apparatus will come in a variety of different shapes and all will use lock-in-place slots with a tapered orifice, a finger orifice in the blade upper section to grip and hold the knife blade securely when replacing them. The knife blades will be positioned between the lock-in-place slots with the tapered orifice sliding against the stationary indexing threaded shaft. The stationary indexing threaded shaft will have left hand threads on one end and right hand threads on the opposite other end so that when the indexing wheel is rotated the inner blade clamp block and the outer blade clamp block will move in unison together or apart. The outer blade clamp block on one side will have calibration markings to establish the location of the inner blade clamp block and the outer blade clamp block. The stationary indexing threaded shaft will be held in place, but can rotate by the means of snap rings on either side of the central portion of the support member. The support member will have cavities on either end with guide sections that the inner blade clamp blocks and the outer blade clamp blocks which translate onto when they are moved back and forth. The pneumatic osteotome mounting rod will be permanently attached to the central portion of the support member.

### BACKGROUND OF THE INVENTION

There is growing need to provide a new and refined method of performing delicate surgical operations including hip, shoulder and knee replacements. The similarity in these operations is that the implants have to be inserted into a major bone in the area and when there is a problem with them, the prostheses has to be removed.

As with any other mechanical device, a total hip replacement can be subject to various forms of mechanical or biological failure. Such a failure may require a revision of the hip replacement to address the cause of failure and its consequences. A revision of a total hip replacement is called a revision or femoral hip stem explant.

The revision hip implant is comprised of four parts that work together to restore the original function of the ball-and-socket joint. These four parts are as follows: (A) a metal hip stem that is inserted into the top of the thighbone; (B) a metal cup which holds the cup liner; (c) a cup liner which holds the femoral head; and (D) the femoral head or ball which is attached to the top of the hip stem and is inserted into the liner to form the ball-and-socket joint.

The wearing down of the plastic component has an unfortunate side effect. The tiny plastic particles that wear off are attacked by your body's immune system, and this immune response also attacks the healthy bone around your implant. This leads to a condition called osteolysis, in which the bone in the area around the joint implant softens as it is absorbed by the body, thus making the implant unstable and in need of revision.

If the bone next to the primary implant is fractured in an accident, revision surgery may be required in order to provide a safe, stable joint. In this case, the original implant may need to be removed, the fracture addressed and a revision joint implanted.

In a low percentage of cases, the hip may become infected after surgery. Although it may be successfully treated with antibiotics, there are severe cases where a follow-up revision surgery may be required.

Hip revision operations are performed relatively infrequently. In the United States, there are approximately 18 revision hip replacements performed for every 100 hip replacements. The most common reasons for revision surgery are as follows: (A) repetitive (recurrent) dislocation of a hip replacement; (B) mechanical failure (implant wear and tear—loosening or breakage); and (C) infection.

Numerous innovations for Joint Revision Surgery Apparatus have been provided in the prior art described as follows. Even though these innovations may be suitable for the specific individual purposes to which they address, they differ from the present Prosthesis Extraction Apparatus hereinafter contrasted. The following is a summary of those prior art patents most relevant to the Prosthesis Extraction Apparatus at hand, as well as a description outlining the difference between the features of the present application and those of the prior art.

U.S. Pat. No. 9,138,242 of Randell J. Lewis describes a femoral hip stem explant system that has an alignment body which is attached to two locations of a femoral bone and has several lockable collet type adjustment features to set the shaft of a reamer or end mill exactly in coincidence with the femoral bone cavity axis. The shaft of the end mill or reamer is supported by a sleeve member, which is inserted into a drill guide central aperture. The drill guide aperture is adjusted first and locked to be in line with the femoral bone cavity. Each of the sleeve members has the same mating outer diameter, which fits into the drill guide central aperture. Accordingly, the sleeve members can be interchanged into the drill guide aperture with shafts of differently sized reamers or end mills.

This patent describes a femoral hip stem explant or revision system that has an alignment body which is attached to two locations of a femoral bone and has several lockable collet type adjustment features to set the shaft of a reamer or end mill exactly in coincidence with the femoral bone cavity axis. This patent describes a very invasive surgery to attach at two locations of a femoral bone where the Prosthesis Extraction Apparatus works from the top of the femur only and leaves the most proximal portion in fairly good shape.

U.S. Pat. No. 6,740,092 of Alan Lombardo et al. describes IM revision tools include reamers with depth markings or stops, an impactor-extractor with a coupling for attaching to tools which are inserted into and removed from the IM canal, a resection guide tower to which a cutting block is attached and which includes a notch which serves as both a witness mark and a holder for a femoral collar, a reversible clean-up cutting block with a quick-connect clamp attachable to the guide tower for resecting the distal femur, a selection of spacer blocks for measuring the space between the femur to determine the size of the components to be installed, a multiple cut cutting guide for preparing the femur, a set of 5 and 10 mm trial wedges, a trial stem valgus adapter, femoral sizing indicators which include indications of anterior/posterior offset, a stabilizer box cutting template which is attachable to the multiple cut cutting guide, and anterior/posterior offset adapters for attaching the femoral component to the IM stem.

This patent describes IM revision tools that include reamers with depth markings or stops, an impactor-extractor with a coupling for attaching to tools which are inserted into and removed from the IM canal, a resection guide tower to which a cutting block is attached. This patent describes a number of tools for positioning and alignment where the Prosthesis Extraction Apparatus uses the trunnion and the sides of the existing prostheses and the proximal portion of the femur for support and alignment of the process.

U.S. Pat. No. 6,258,095 of Alan Lombardo et al. describes IM revision tools include reamers with depth markings or stops, an impactor-extractor with a coupling for attaching to tools which are inserted into and removed from the IM canal, a resection guide tower to which a cutting block is attached and which includes a notch which serves as both a witness mark and a holder for a femoral or tibial collar, a reversible clean-up cutting block with a quick-connect clamp attachable to the guide tower for resecting the distal femur and separate left and right clean-up cutting blocks for resecting the proximal tibia, a selection of spacer blocks for measuring the space between the tibia and femur to determine the size of the components to be installed, a multiple cut cutting guide for preparing the femur, a set of 5 and 10 mm trial wedges, a trial stem valgus adapter, femoral sizing indicators which include indications of anterior/posterior offset, a stabilizer box cutting template which is attachable to the multiple cut cutting guide, and anterior/posterior offset adapters for attaching the femoral component to the IM stem. The tools according to the invention are modular and can also be used in primary knee arthroplasty without IM fixation. The methods of the invention provide accurate location of bone cuts so that the revision prosthetic is correctly oriented relative the IM canal and the bone cuts.

This is another patent that describes a number of tools for positioning and alignment where the Prosthesis Extraction Apparatus uses the trunnion and the sides of the existing prostheses and the proximal portion of the femur for support and alignment of the process.

US Patent Application Publication No. 2014/0371750 of Greg Klein et al. describes an apparatus for removal of a femoral implant that may include a handle portion and a cutting blade opposite the handle portion. The cutting blade may include a cutting edge, wherein the cutting edge includes a non-linear shape to substantially match at least a portion of a profile of the femoral implant to be removed.

This patent describes an apparatus for removal of a femoral implant that may include a handle portion and a cutting blade opposite the handle portion but does not describe the unique features of the Prosthesis Extraction Apparatus

U.S. Pat. No. 9,282,981 of Rebecca L. Chaney describes a number of orthopedic surgical instruments for use in a surgical procedure to prepare a patient's femur to receive an orthopedic prosthesis are disclosed. The tools include guide tools, cutting tools, surgical blocks, and other orthopedic surgical instruments configured to plan and guide the preparation of the patient's femur.

This patent describes a number of orthopedic surgical instruments for use in a surgical procedure to prepare a patient's femur to receive an orthopedic prosthesis but does not describe unique features of the Prosthesis Extraction Apparatus.

None of the foregoing prior art teaches or suggests the particular unique features of the Joint Revision Surgery Apparatus and thus clarifies the need for further improvements in the devices that can be used for these purposes.

In this respect, before explaining at least one embodiment of the Prosthesis Extraction Apparatus detail it is to be understood that the design is not limited in its application to the details of construction and to the arrangement of the components set forth in the following description or illustrated in the drawings. The Prosthesis Extraction Apparatus are capable of other embodiments and of being practiced and carried out in various ways. Also, it is to be understood that the phraseology and terminology employed herein are for the purpose of description and should not be regarded as limiting.

### SUMMARY OF THE INVENTION

The principle advantage of the of the Prosthesis Extraction Apparatus is to have safer and more successful joint revision surgery.

Another advantage of using the Prosthesis Extraction Apparatus is to have significantly less blood loss during the procedure.

Another advantage of using the Prosthesis Extraction Apparatus is to have much shorter operating room times during the procedure.

Another advantage of using the Prosthesis Extraction Apparatus is to have less instrumentation required, that is fewer cables and long stems during the procedure.

Another advantage of using the Prosthesis Extraction Apparatus is to have quicker times to have full weight bearing capability after surgery.

Another advantage of using the Prosthesis Extraction Apparatus is to have less complications.

Another advantage of using the Prosthesis Extraction Apparatus is to have less mortality.

Another advantage of using the Prosthesis Extraction Apparatus is to have less morbidity.

An advantage of the first alternate embodiment of the Prosthesis Extraction Apparatus is to have the internal elongated cavity allowing the upward movement of the existing prostheses.

An advantage of the first alternate embodiment of the Prosthesis Extraction Apparatus is that without using the Pneumatic Osteotome a small hammer can be used to manually control and tap on the end of the mounting rod connected to the support member.

Another advantage of using the first alternate embodiment of the Prosthesis Extraction Apparatus is by rotating the indexing wheel the two blades can be moved equally together or away from the sides of the prosthesis.

Another advantage of using the first alternate embodiment of the Prosthesis Extraction Apparatus is by rotating the indexing wheel the two blades can be moved together or back by the means of the left and right hand threads on either end of the stationary indexing wheel threaded shaft held stationary within the support member.

Another advantage of using the first alternate embodiment of the Prosthesis Extraction Apparatus is that the stationary indexing wheel threaded shaft is held stationary but can still rotate by the means of snap rings at either end of the support member central member.

Another advantage of using the first alternate embodiment of the Prosthesis Extraction Apparatus is that the calibration marking on the upper surface of the support member indicates the blades location and their intended movement together or back away from the prosthesis.

Another advantage of using the first alternate embodiment of the Prosthesis Extraction Apparatus is the blades can easily be replaced by backing off on the indexing wheel and moving the blades to the side.

Another advantage of using the first alternate embodiment of the Prosthesis Extraction Apparatus is the having the sharp edges of the blades in a controlled position away from the surfaces of the prosthesis, eliminating the scraping metal particles into the wound.

An advantage of the second alternate embodiment of the Prosthesis Extraction Apparatus is having a simpler device to perform a similar process for other Joint Revision Surgeries.

Another advantage of the second alternate embodiment of the Prosthesis Extraction Apparatus is that the blades can be moved independently against or away from the prosthesis.

Another advantage of the third alternate embodiment of the Prosthesis Extraction Apparatus is that it can be less expensive to manufacture.

Another advantage of the third alternate embodiment of the Prosthesis Extraction Apparatus is the knife blades can be replaced by sliding out of the side of the support member and a replacement knife blade inserted with the locking sections locking knife blades in the cavities in the slots of the support members.

Another advantage of the fourth alternate embodiment of the Prosthesis Extraction Apparatus is to have another simpler device to perform the surgery having the Apparatus with the with the blades secured permanently in place in the base member.

Finally, an advantage of the fifth alternate embodiment of the Prosthesis Extraction Apparatus is that it can be constructed using a gear box having gears to actuate the alignment and working width of the knife blades.

The Joint Revision Surgery using the Prosthesis Extraction Apparatus process entails the mounting of an alignment member to the prosthesis with a screw or barbed mounting pin within a threaded hole in the upper surface of the prosthesis.

The pneumatic osteotome with the chuck connected to the mounting rod of the Prosthesis Extraction Apparatus is positioned with the alignment member inserted in the guide slot in the edge of the Apparatus and the device is lowered with the two blades against the top sides of the prosthesis.

The Prosthesis Extraction Apparatus process entails the positioning of the Apparatus over the trunnion end of the existing prostheses within its internal cavity. The knife blades will additionally be used to properly align the device with the side surfaces of the existing prostheses.

The knife blades will be used to loosen both sides of the existing prostheses with the blades used with the sharp edge section away from the side surfaces of the existing prostheses to keep the knives from digging into or scraping the metal.

With respect to the above description then, it is to be realized that the optimum dimensional relationships for the parts of the Prosthesis Extraction Apparatus, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present design. Therefore, the foregoing is considered as illustrative only of the principles of the Prosthesis Extraction Apparatus. Further, since numerous modifications and changes will readily occur to those skilled in the art, it is not desired to limit the Prosthesis Extraction Apparatus to the exact construction and operation shown and described, and accordingly, all suitable modifications and equivalents may be resorted to, falling within the scope of this application.

While the apparatus and method has or will be described for the sake of grammatical fluidity with functional explanations, it is to be expressly understood that the claims, unless expressly formulated under 35 USC 112, or similar applicable law, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112, or similar applicable law. The Prosthesis Extraction Apparatus can be better visualized by turning now to the following drawings wherein like elements are referenced by like numerals.

### BRIEF DESCRIPTION OF THE DRAWINGS

The accompanying drawings, which are incorporated in and form a part of this specification, illustrate embodiments of the Joint Revision Surgery Apparatus and together with the description, serve to explain the principles of this application.

FIG. 1 depicts an exploded perspective of the preferred embodiment of the Prosthesis Extraction Apparatus with the two knife blades moved out from the side of the support member.

FIG. 2 depicts an end view of the preferred embodiment of the Prosthesis Extraction Apparatus support member with the indexing wheel.

FIG. 3A depicts a top plan view of the preferred embodiment of the Prosthesis Extraction Apparatus in the extended position with the alignment orifice on the upper side of the support member.

FIG. 3B depicts an end view of the reverse side of the indexing wheel for use with the Prosthesis Extraction Apparatus support member with the indexing wheel including calibration indentions.

FIG. 4 depicts a cross section view through the side of the preferred embodiment of the Prosthesis Extraction Apparatus in the extended position.

FIG. 5 depicts a top view of the preferred embodiment of the Prosthesis Extraction Apparatus 10A in the compressed position to show the positions of the outer blade clamp blocks.

FIG. 6 depicts a cross section view through the side of the preferred embodiment of the Prosthesis Extraction Apparatus in the compressed position to further clarify the positions of the outer blade clamp blocks.

FIG. 7 depicts a partially sectioned end view of the preferred embodiment of the Prosthesis Extraction Apparatus with one of the blades in place.

FIG. 8 depicts a partially sectioned view of the preferred embodiment of the Prosthesis Extraction Apparatus depicts the indexing threaded shaft.

FIG. 9 depicts a cross section through one of the knife blades between the inner blade clamp block and outer blade clamp block.

FIG. 10 depicts an edge view of a curved surface of the knife blade.

FIG. 11 depicts a plan view of a knife blade used in the Prosthesis Extraction Apparatus.

FIG. 12 depicts an edge view of a curved knife blade.

FIG. 13 depicts a plan view of a knife blade with inward curved knife blade edges.

FIG. 14 depicts an edge view of a concaved knife blade.

FIG. 15 depicts a plan view of a concaved curved knife blade with outward curved knife blade edges.

FIG. 16 depicts a plan view of an angled cut knife blade.

FIG. 17 depicts a plan view of an extended angled cut knife blade.

FIG. 18 depicts a side view of the procedure where the pneumatic osteotome, osteotome chuck and the osteotome mounting rod are connected to the support member.

FIG. 19 depicts a front view of the Prosthesis Extraction Apparatus with a typical prosthesis between two of the knife blades.

FIG. 20 depicts a side view of the Prosthesis Extraction Apparatus with a typical prosthesis between two of the knife blades are connected to the support member.

FIG. 21 depicts a side view of the Prosthesis Extraction Apparatus alignment bar.

FIG. 22 depicts a front view of the Prosthesis Extraction Apparatus alignment bar with an orifice in the angled section.

FIG. 23 depicts a side view cross section of the second alternate embodiment of the Prosthesis Extraction Apparatus with a square support member having square chambers and at either end.

FIG. 24 depicts a side view cross section of the second alternate embodiment of the Prosthesis Extraction Apparatus support member having square chambers at either end.

FIG. 25 depicts an end view of the second alternate embodiment of the Prosthesis Extraction Apparatus support member.

FIG. 26 depicts front and a side views of the inner blade clamping block of the second alternate embodiment of the Prosthesis Extraction Apparatus.

FIG. 27 depicts front and side views of the outer blade clamping block of the second alternate embodiment of the Prosthesis Extraction Apparatus

FIG. 28 depicts the side view of the knife blade with the tapered orifice between the square inner blade clamp block and the square threaded outer blade clamp block.

FIG. 29 depicts a perspective view of the third embodiment of the Prosthesis Extraction Apparatus with T-topped knife blade blades.

FIG. 30 depicts a top view of the third embodiment of the Prosthesis Extraction Apparatus with the rotating locking plate.

FIG. 31 depicts a first side view of the third embodiment of the Prosthesis Extraction Apparatus.

FIG. 32 depicts a second side view of the third embodiment of the Prosthesis Extraction Apparatus with one of the knife blades partially removed and the other in place.

FIG. 33 depicts the bottom view of the third embodiment of the Prosthesis Extraction Apparatus.

FIG. 34 depicts a perspective view of the fourth embodiment of the Prosthesis Extraction Apparatus.

FIG. 35 depicts a top view of the fourth embodiment of the Prosthesis Extraction Apparatus.

FIG. 36 depicts a first side view of the fourth embodiment of the Prosthesis Extraction Apparatus.

FIG. 37 depicts a bottom view of the fourth embodiment of the Prosthesis Extraction Apparatus with only one of the knife blades in place, illustrating that the apparatus may be used with only one surgical knife blade.

FIG. 38 depicts the bottom view of the fourth embodiment of the Prosthesis Extraction Apparatus.

FIG. 39 depicts a front partial cross-sectional view of the fifth embodiment of the Prosthesis Extraction Apparatus, illustrating a gearbox blade adjustment actuation mechanism.

FIG. 40 depicts an enlarged front partial cross-sectional view of the fifth embodiment of the Prosthesis Extraction Apparatus, illustrating the gearbox actuation mechanism from FIG. 39.

FIG. 41 depicts a partial top and front perspective view of a sixth embodiment of the Prosthesis Extraction Apparatus surgical knife blade mounting and support block having a two-piece adjustable H-block with a threaded rod and single adjustment knob.

FIG. 42 depicts a top view of a sixth embodiment of the Prosthesis Extraction Apparatus surgical knife blade mounting and support block shown in FIG. 41 having a two-piece adjustable H-block with a threaded rod and single adjustment knob.

FIG. 43 depicts a partial cross-sectional view of a sixth embodiment of the Prosthesis Extraction Apparatus surgical knife blade mounting and support block shown in FIG. 41 FIG. 42 having a two-piece adjustable H-block with a threaded rod and single adjustment knob.

FIG. 44 depicts a side elevational view of a sixth embodiment of the Prosthesis Extraction Apparatus surgical knife blade mounting and support block shown in FIG. 41 having a two-piece adjustable H-block with a threaded rod and single adjustment knob.

FIG. 45 depicts a knife blade with a threaded nut for attachment to the sixth embodiment of the Prosthesis Extraction Apparatus surgical knife blade mounting and support block having a two-piece adjustable H-block with a threaded rod and single adjustment knob.

FIG. 46 depicts a knife blade with a threaded nut for attachment to the sixth embodiment of the Prosthesis Extraction Apparatus surgical knife blade mounting and support block having a two-piece adjustable H-block with a threaded rod and single adjustment knob.

FIG. 47 depicts a knife blade with a threaded nut for attachment to the sixth embodiment of the Prosthesis Extraction Apparatus surgical knife blade mounting and support block having a two-piece adjustable H-block with a threaded rod and single adjustment knob.

FIG. 48 depicts a knife blade with a threaded nut for attachment to the sixth embodiment of the Prosthesis Extraction Apparatus surgical knife blade mounting and support block having a two-piece adjustable H-block with a threaded rod and single adjustment knob.

### DETAILED DESCRIPTION OF THE PREFERRED EMBODIMENTS

As required, the detailed embodiments of the present Prosthesis Extraction Apparatus 10A, 10B, 10C, 10D, 10E and 10F are disclosed herein, however, it is to be understood that the disclosed embodiments are merely exemplary of the design that may be embodied in various forms. Therefore, specific functional and structural details disclosed herein are not to be interpreted as limiting, but merely as basic for the claims and as a representative basis for teaching one skilled in the art to variously employ the present design in virtually any appropriately detailed structure.

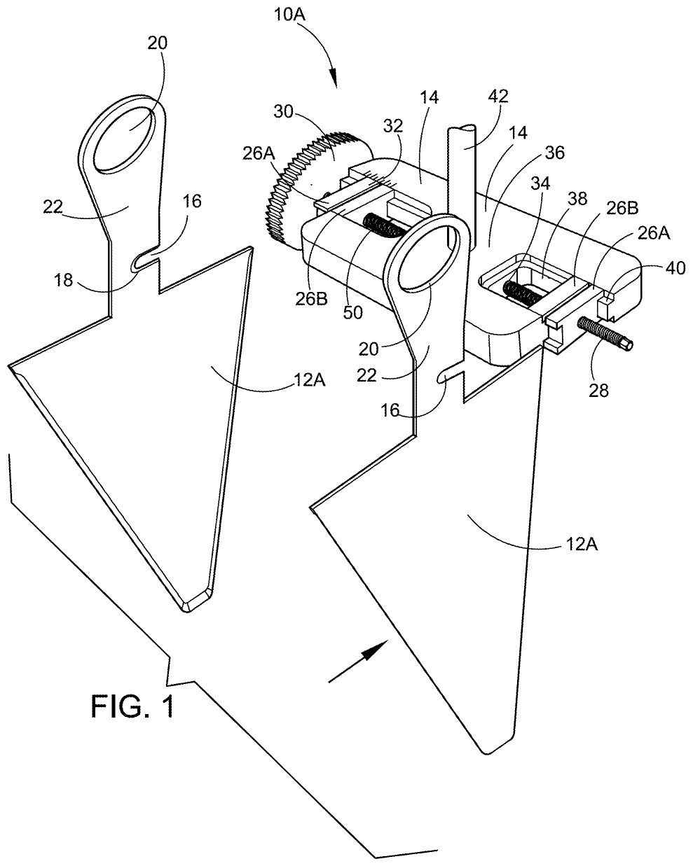

FIG. 1 depicts an exploded perspective of the preferred embodiment of the Prosthesis Extraction Apparatus 10A with the two knife blades 12 moved out from the side of the support member 14. The knife blades 12 used additionally on the Apparatus 10A and 10B can come in a variety of different shapes and all will use the lock-in-place slots 16 with the tapered orifice 18. A finger orifice 20 in the blade upper section 22 is to grip and hold the knife blade 12 securely when replacing them. The knife blades 12 are positioned between the lock-in-place slots 16 with the tapered orifice 18 slide against the stationary indexing threaded shaft 28. The stationary indexing threaded shaft 28 has left hand threads on one end and right hand threads on the other so that when the indexing wheel 30 is rotated the inner blade clamp block 24 and the outer blade clamp block 26 will move together or apart. The outer blade clamp block 24 on one side will have calibration markings 32 to establish the location of the inner blade clamp block 24 and the outer blade clamp block 26. The stationary indexing threaded shaft 28 is held in place, but can rotate by the means of snap rings 34 on either side of the central portion 36 of the support member 14. The support member 14 has cavities 38 on either end with guide sections 40 that the inner blade clamp blocks 24 and the outer blade clamp blocks 26 which translate onto when they are moved back and forth. The pneumatic osteotome mounting rod 42 is permanently attached to the central portion 36 of the support member 14.

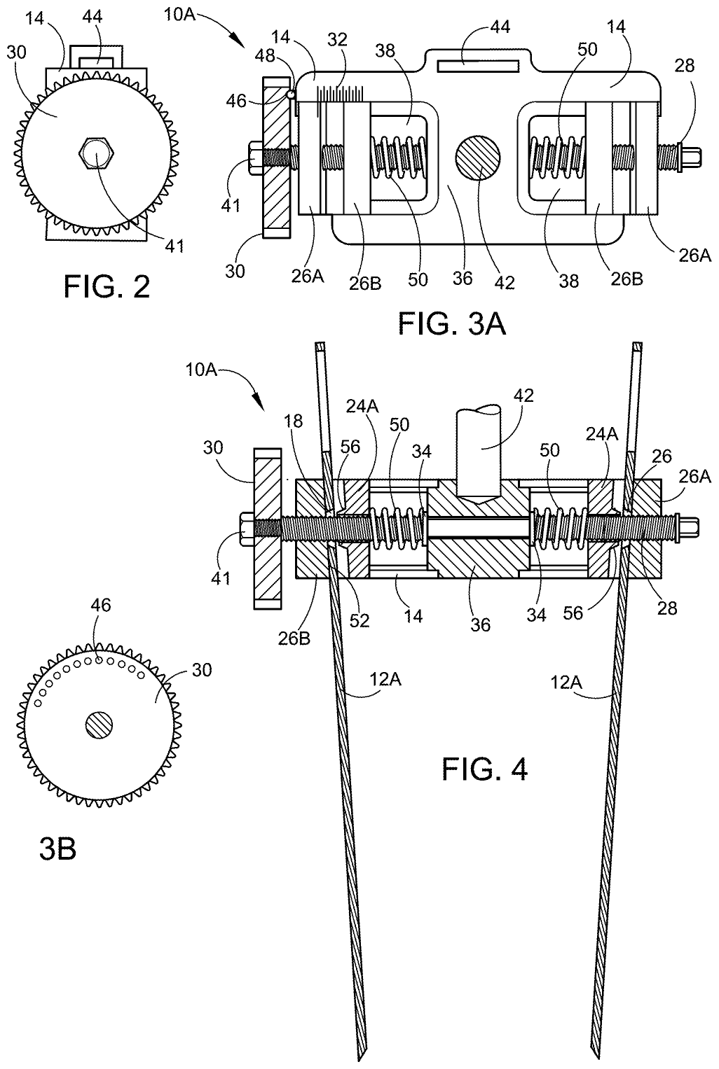

FIG. 2 depicts an end view of the preferred embodiment of the Prosthesis Extraction Apparatus 10A support member 14 with the indexing wheel 30 locked on the end of the indexing threaded shaft 28 by the means of a conventional hex nut 41. The alignment orifice 44 on the upper side of the support member 14 is used for support and alignment with the prosthesis.

FIG. 3A depicts a top plan view of the preferred embodiment of the Prosthesis Extraction Apparatus 10A in the extended position with the alignment orifice 44 on the upper side of the support member 14. When the threaded outer blade clamp blocks 26 and inner blade clamp blocks 24 are in these locations the knife blades 12 can be inserted or removed. The indexing wheel 30 is shown in cross section to display the polarity of calibrating indentions 46 and the mating spring-loaded ball detent 48. When the indexing wheel 30 is rotated the threaded outer blade clamp blocks 26 compresses the blades against the inner blade clamp blocks 24, which then compresses the springs 50 against the support member 14 central portion 36 to achieve the desired locations of the two knife blades 12.

FIG. 3B depicts an inner view of the indexing wheel 30 to expose the locations of the polarity of calibrating indentions 46 on the side adjacent to the support member 14.

FIG. 4 depicts a cross section view through the side of the preferred embodiment of the Prosthesis Extraction Apparatus 10A in the extended position to clarify the positions of the outer blade clamp blocks 26 with the flat surfaces 52 against the knife blades 12. The knife blades 12 have a concave orifice 18 with the lock-in-place slots 16. The inner blade clamp blocks 24 have tapered mating sections 56 on the surfaces that mate with the tapered orifice 18 and the lock-in-place slots 16 on the knife blades 12 securing the blades in place. The clearance hole through the inner blade clamp block 24 allow it to slide when compressed by the means of the outer blade clamp blocks 26 moving on the threads of the indexing threaded shaft 28 to compresses the springs 50. This process is used to move the knife blades in place.

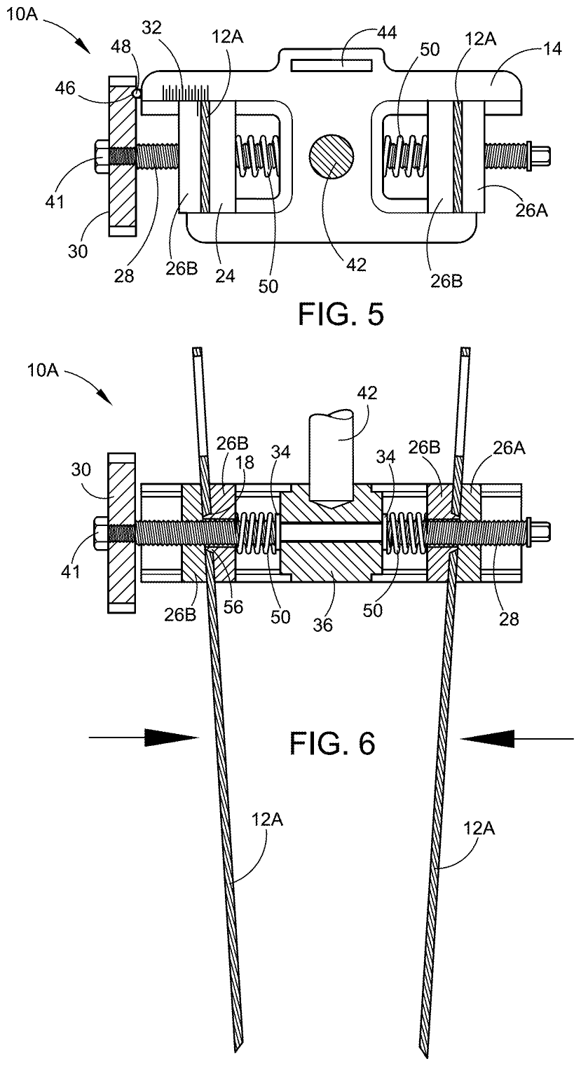

FIG. 5 depicts a top view of the preferred embodiment of the Prosthesis Extraction Apparatus 10A in the compressed position to show the positions of the outer blade clamp blocks 26 with the flat surfaces 52 against the knife blades 12. The knife blades 12 have concave orifice 18 with the lock-in-place slots 16. The inner blade clamp blocks 24 have tapered mating sections 56 on the surfaces that mate with the tapered orifice 18 and the lock-in-place slots 16 on the knife blades 12 securing the blades in place. The clearance hole through the inner blade clamp block 24 allow it to slide when compressed by the means of the outer blade clamp blocks 26 moving on the threads of the indexing threaded shaft 28 to compresses the springs 50. This process is used to move the knife blades in place.

FIG. 6 depicts a cross section view through the side of the preferred embodiment of the Prosthesis Extraction Apparatus 10A in the compressed position to further clarify the positions of the outer blade clamp blocks 26 with the flat surfaces 52 against the knife blades 12. The knife blades 12 have a concave orifice 18 with the lock-in-place slots 16. The inner blade clamp blocks 24 have tapered mating sections 56 on the surfaces that mate with the tapered orifice 18 and the lock-in-place slots 16 on the knife blades 12 securing the blades in place. The clearance hole through the inner blade clamp block 24 allow it to slide when compressed by the means of the outer blade clamp blocks 26 moving on the threads of the indexing threaded shaft 28 to compresses the springs 50. This process is used to move the knife blades in place.

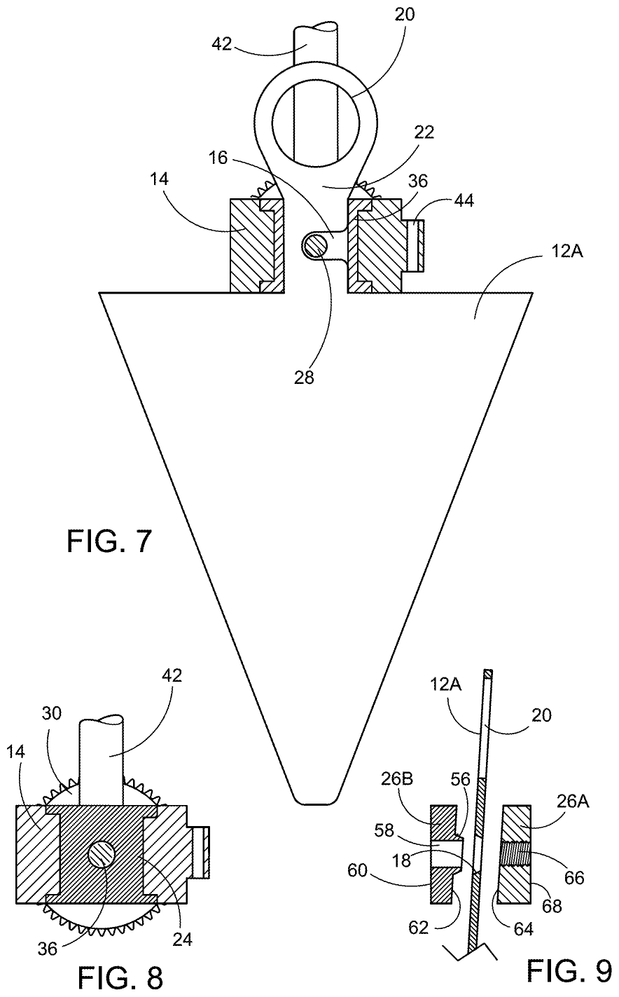

FIG. 7 depicts a partially sectioned end view of the preferred embodiment of the Prosthesis Extraction Apparatus 10A with one of the knife blades 12A in place with the blade upper section 22 and the finger orifice 20. The indexing threaded shaft 28 supported by the support member 14 extends through the lock-in-place slot 16.

FIG. 8 depicts a partially sectioned view of the preferred embodiment of the Prosthesis Extraction Apparatus 10A depicts the indexing threaded shaft 28 extending through the blade clamp block 24 from the indexing wheel 30.

FIG. 9 depicts a cross section through one of the knife blades 12A between the inner blade clamp block 26B and outer blade clamp block 26A in the open position. The inner blade clamp block 26B has a smooth hole 58 through the center of the flat vertical surface 60 on one side and a ten degree angled surface 62 with a tapered mating section 56 matching the tapered orifice portion 18, of the lock-in-place slot 16 in the knife blade 12A. The outer blade clamp block 26A has a ten degree angled flat surface 64 with a left or right hand threaded orifice 66 on the vertical surface 68 to match the left and right hand threads on the indexing threaded shaft 28.

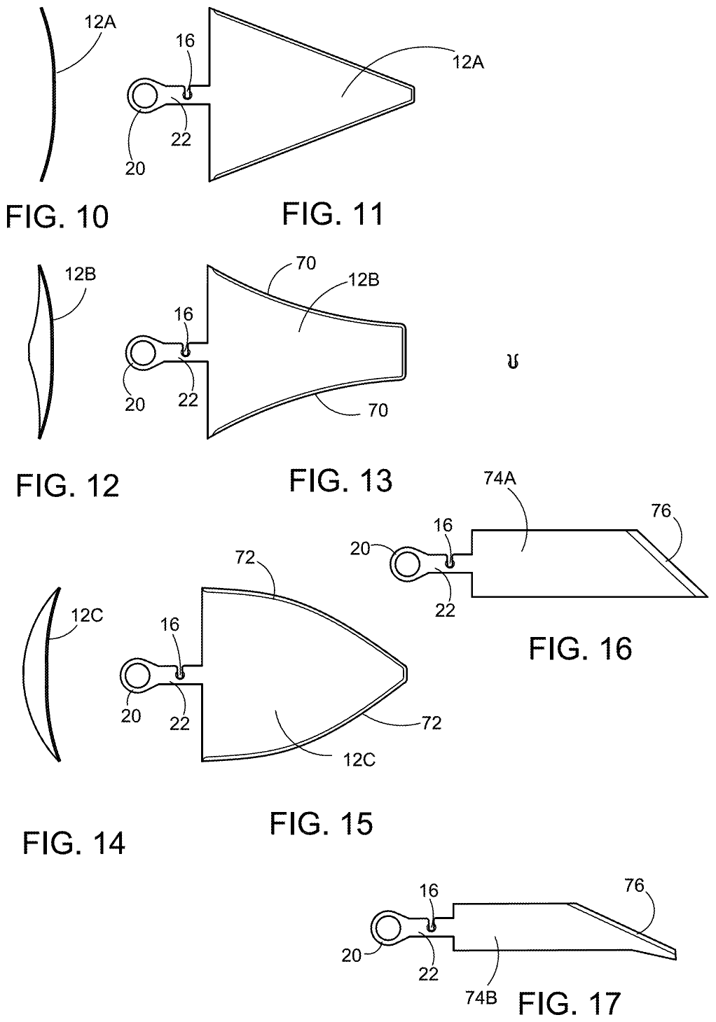

FIG. 10 depicts an edge view of a curved surface 68 of the knife blade 12A used in the Prosthesis Extraction Apparatus. All knife blades will have a finger orifice 20 on the blade upper section 22 and a lock-in-place slot 16.

FIG. 11 depicts a plan view of a knife blade 12A used in the Prosthesis Extraction Surgery.

FIG. 12 depicts an edge view of a curved knife blade 12B used in the Prosthesis Extraction Apparatus.

FIG. 13 depicts a plan view of a knife blade 12B with inward curved knife blade edges 70 used in the Prosthesis Extraction Apparatus.

FIG. 14 depicts an edge view of a concaved knife blade 12C used in the Prosthesis Extraction Apparatus.

FIG. 15 depicts a plan view of a concaved curved knife blade 12C with outward curved knife blade edges used in the Prosthesis Extraction Apparatus.

FIG. 16 depicts a plan view of an angled cut knife blade 74A with an angled cutting edge 76 with the beveled sharp edge away from the prosthesis metal surface.

FIG. 17 depicts a plan view of an extended angled cut knife blade 74B with an extended angle cutting edge 78 with the beveled sharp edge away from the prosthesis metal surface.

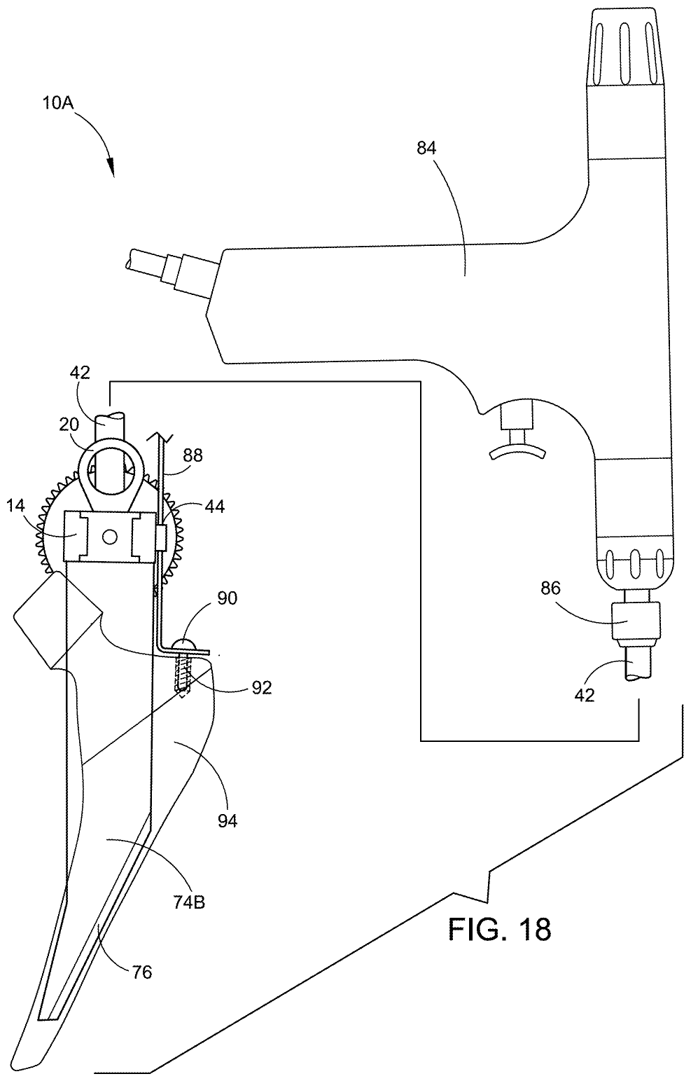

FIG. 18 depicts a side view of the procedure where the pneumatic osteotome 84, osteotome chuck 86 and the osteotome mounting rod 42 are connected to the support member 14 with the alignment bar 88 going through the alignment orifice 44 and connected by the means of a screw or barbed stem attachment 90 into a threaded hole 92 in the top surface in the prosthesis 94. The angled cut knife blade 74A with an extended angle cutting edge 76 with the beveled sharp edge away from the prosthesis metal surface is illustrated in front of the prosthesis 94.

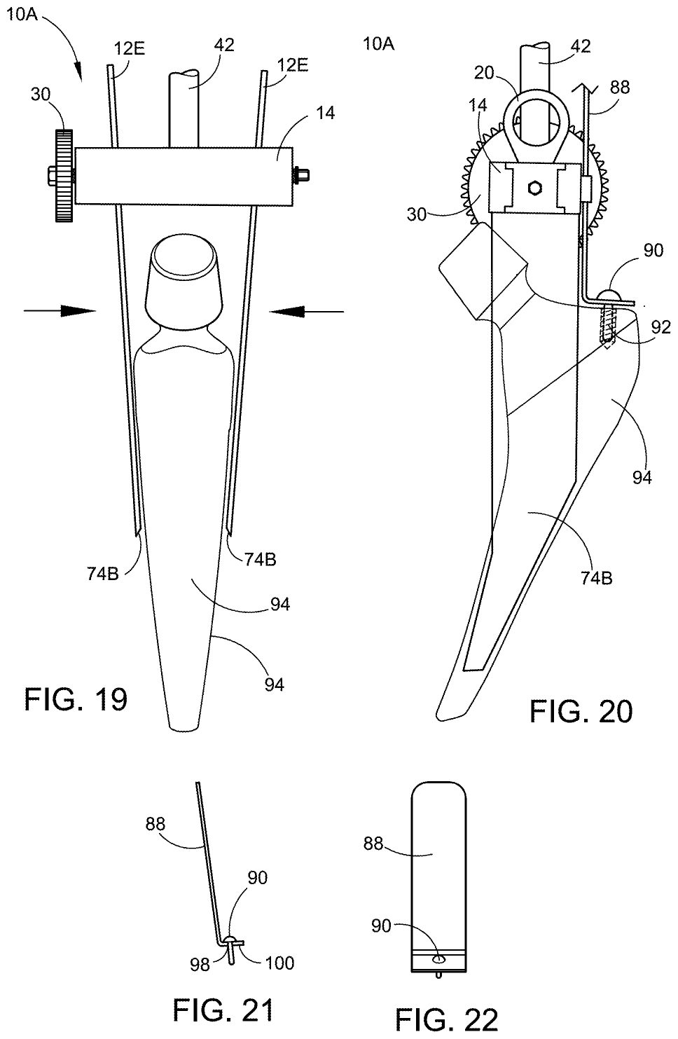

FIG. 19 depicts a front view of the Prosthesis Extraction Apparatus 10A with a typical prosthesis 94 between two of the knife blades 12E with the indexing wheel 30 on the support member 14 supported by the means of the osteotome mounting rod 42.

FIG. 20 depicts a side view of the Prosthesis Extraction Apparatus 10A with a typical prosthesis between two of the knife blades 12E are connected to the support member 14 with the alignment bar 88 going through the alignment orifice 44 and connected by the means of a screw or barbed stem attachment 90 into a threaded hole 92 in the top surface in the prosthesis 94. The conventional knife blade 74 with an extended angle cutting edge 78 is illustrated in front of the prosthesis 94.

FIG. 21 depicts a side view of the Prosthesis Extraction Apparatus 10A alignment bar 88 with an orifice 98 in the angled section 100 for the screw or barbed stem attachment 90 to means to have the Prosthesis Extraction Apparatus 10A be able to slide up or down and still have the control over the prosthesis 94.

FIG. 22 depicts a front view of the Prosthesis Extraction Apparatus 10A alignment bar 88 with an orifice 98 in the angled section 100 for the screw or barbed stem attachment 90 to the prosthesis 94.

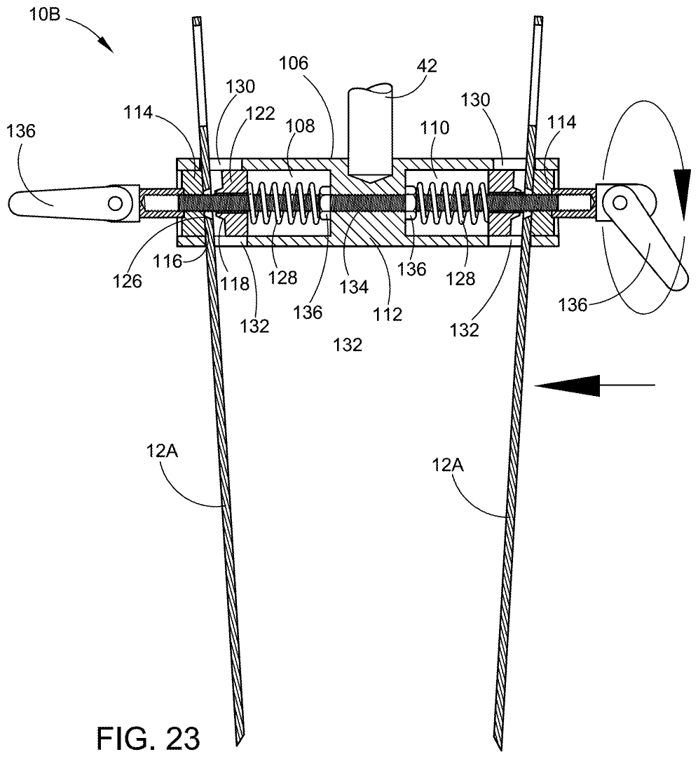

FIG. 23 depicts a side view cross section of the second alternate embodiment of the Prosthesis Extraction Apparatus 10B with a square support member 106 having square chambers 108 and 110 at either end of the threaded center section 112. Square threaded outer blade clamp blocks 114 with the flat surfaces 116 are against the knife blades 12A. The knife blades 12A have a tapered orifice 118 with the lock-in-place slots 18. The inner blade clamp blocks 122 have tapered mating sections 124 on the surfaces that mate with the tapered orifice 126 and the lock-in-place slots 120 on the knife blades 12A securing the blades in place. The clearance hole through the square inner blade clamp block 122 allow them to slide when compressed by the means for the square outer blade clamp blocks 114 to compresses the springs 128. The square support member 106 has openings 130 on the top and openings 132 on the bottom for the knife blades 12A. The all-threaded center shaft 134 is locked in place by the means of two hex nuts 136 and the threaded swivel adjustment members 138 on each end so that the knife blades 12A can be moved in and out separately.

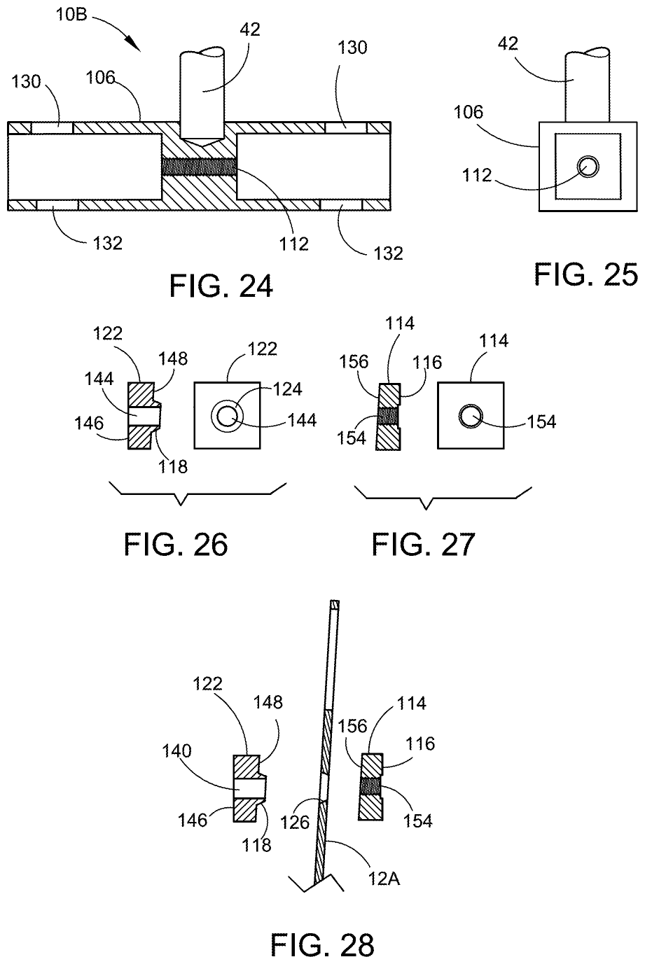

FIG. 24 depicts a side view cross section of the second alternate embodiment of the Prosthesis Extraction Apparatus 10B support member 106 having square chambers 108 and 110 at either end of the threaded center section 112. The osteotome mounting rod 42 is mounted in the center of the top surface.

FIG. 25 depicts an end view of the second alternate embodiment of the Prosthesis Extraction Apparatus 10B support member 106.

FIG. 26 depicts front and a side views of the inner blade clamping block 122 of the second alternate embodiment of the Prosthesis Extraction Apparatus 106 that has a smooth hole 144 through the center of the flat vertical surface 146 on one side and a ten degree angled surface 148 with a tapered conical section 118 on the surface.

FIG. 27 depicts front and a side views of the outer blade clamping block 114 of the second alternate embodiment of the Prosthesis Extraction Apparatus 10B that has a ten degree angled flat surface 156 with a threaded orifice 154 off of the vertical surface 116.

FIG. 28 depicts the side view of the knife blade 12A with the tapered orifice 126 between the square inner blade clamp block 122 and the square threaded outer blade clamp block 114 of the second alternate embodiment of the Prosthesis Extraction Apparatus 10B. The inner blade clamping block 122 has a smooth hole 140 through the center of the flat vertical surface 146 on one side and a ten degree angled surface 148 with a tapered conical section 118 on the surface. The outer blade clamping block 114 has a ten degree angled flat surface 156 with a threaded orifice 154 off of the vertical surface 116.

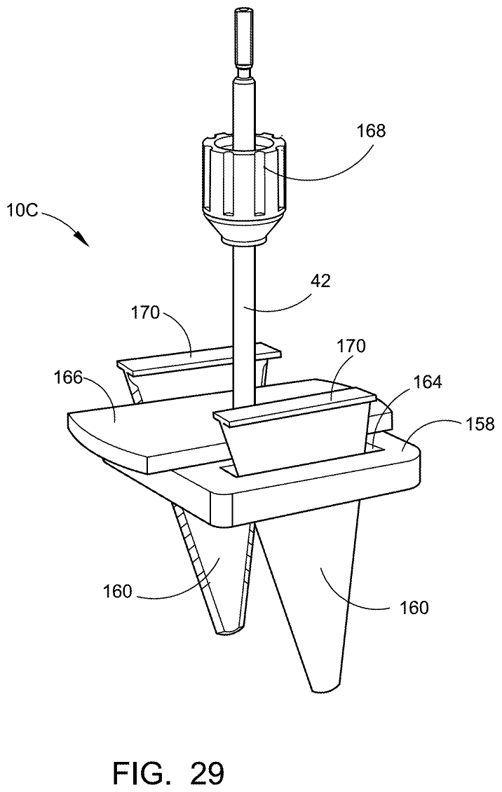

FIG. 29 depicts a perspective view of the third embodiment of the Prosthesis Extraction Apparatus 10C with T-topped knife blade blades 160 that extend through 10 degree slots 162 in the blade housing block 158 with a rectangular cavities 164 matching the “T” portion of the T-topped knife blades 160. After the T-topped knife blade blades 160 are inserted into the 10 degree slots 162 of the rectangular cavities 164 the rotating locking member 166 is rotated on the osteotome mounting rod 42. The osteotome chuck 168 is shown on the osteotome mounting rod 42 with the rotating locking member 166 covering the T-topped knife blades 160 and securing their “T” section into the rectangular cavities 164.

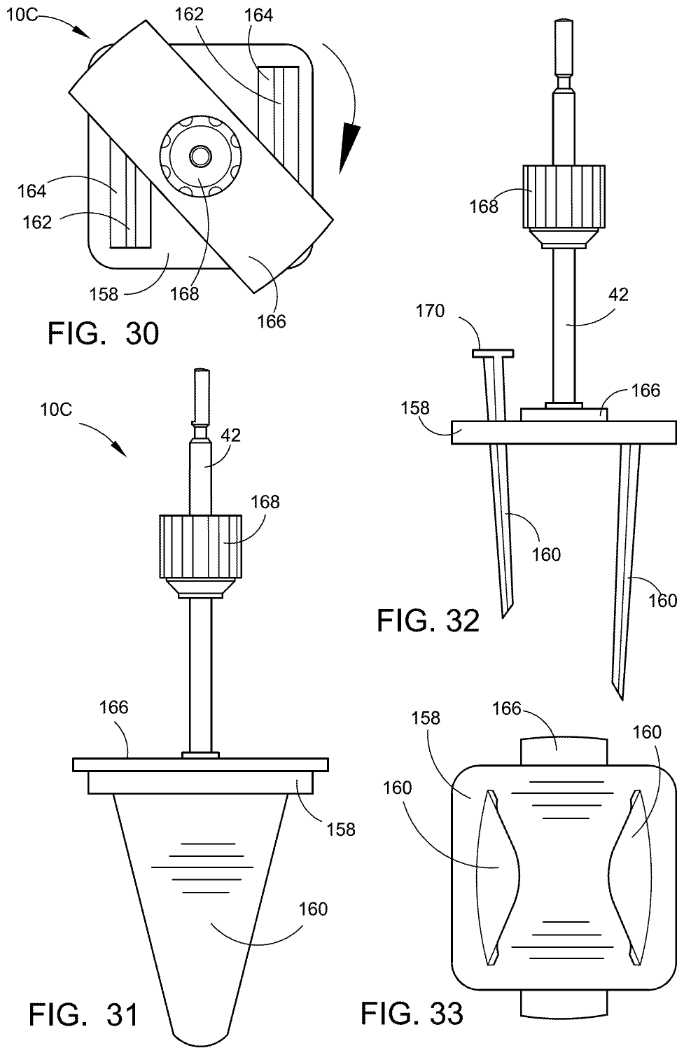

FIG. 30 depicts a top view of the third embodiment of the Prosthesis Extraction Apparatus 10C with the rotating locking member 166 partially covering the top of the blade housing block 158 with the knife blade slots 162 and the rectangular cavities 164 exposed.

FIG. 31 depicts a first side view of the third embodiment of the Prosthesis Extraction Apparatus 10C showing the osteotome mounting rod 42 with the osteotome chuck 168. The rotating locking member 166 is on top of the blade housing block 158 with one of the T-topped knife blades 160 exposed.

FIG. 32 depicts a second side view of the third embodiment of the Prosthesis Extraction Apparatus 10C showing the osteotome mounting rod 42 with the osteotome chuck 168 with one of the T-topped knife blades 160 partially removed and the other in place.

FIG. 33 depicts the bottom view of the third embodiment of the Prosthesis Extraction Apparatus 10C with the bottom of the T-topped knife blades 160 exposed and the rotating locking member 166 behind the blade housing block 158.

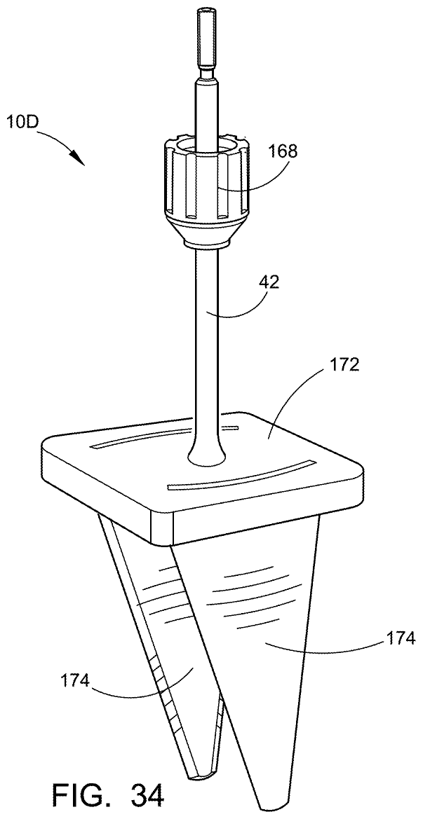

FIG. 34 depicts a perspective view of the fourth embodiment of the Prosthesis Extraction Apparatus 10D with the osteotome mounting rod 42 and the osteotome chuck 168 attached to the blade mounting block 172. The flat toped knife blades 174 are permanently attached to the blade mounting block 172 with each at a 10 degree inward angle.

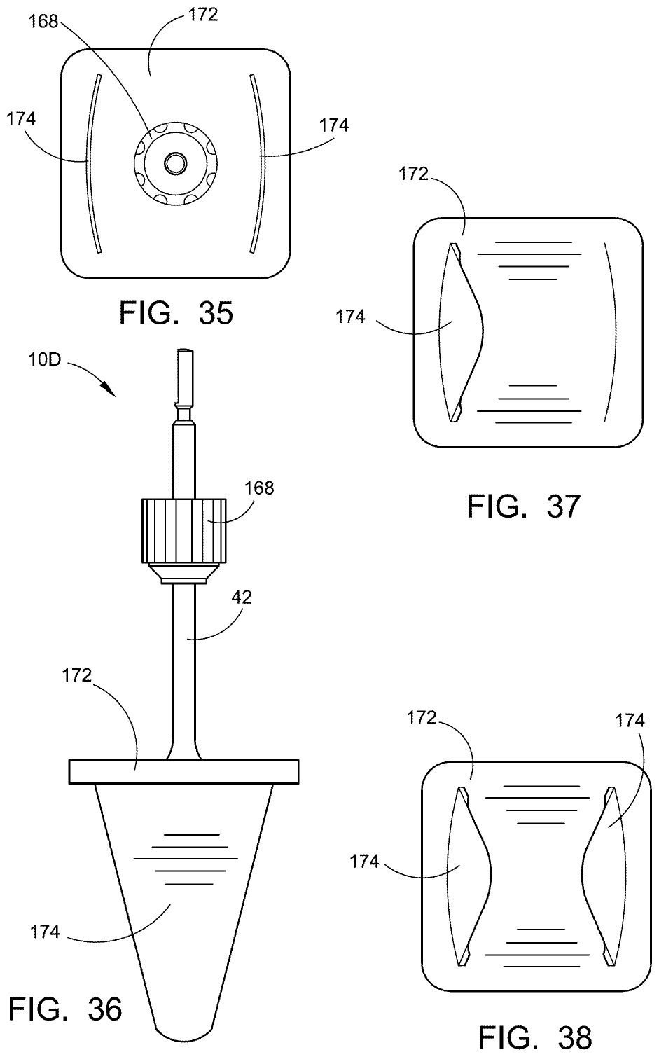

FIG. 35 depicts a top view of the fourth embodiment of the Prosthesis Extraction Apparatus 10D with the flat topped knife blades 174 permanently attached to the blade mounting block 172 having the top of the osteotome chuck 168 showing.

FIG. 36 depicts a first side view of the fourth embodiment of the Prosthesis Extraction Apparatus 10D with one of the flat topped knife blades 174 showing permanently attached to the blade mounting block 172 with the osteotome mounting rod 42 and the osteotome chuck 168 above.

FIG. 37 depicts a bottom view of the fourth embodiment of the Prosthesis Extraction Apparatus 10D with one of the surgical knife blades 174 permanently attached to the blade mounting block 172, illustrating that the Prosthesis Extraction Apparatus 10D made be used with only one of the knife blades 174 permanently attached to the blade mounting block 172.

FIG. 38 depicts the bottom view of the fourth embodiment of the Prosthesis Extraction Apparatus 10D with both of the surgical knife blades 174 permanently attached to the blade mounting block 172.

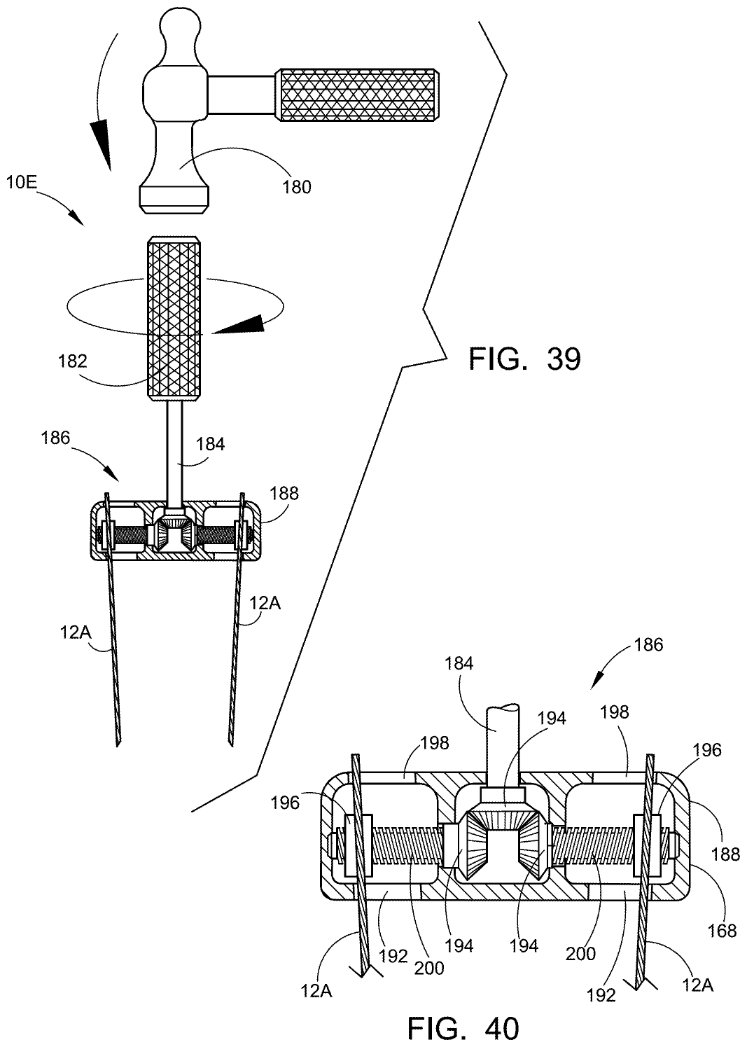

FIG. 39 depicts a front partial cross-sectional view of the fifth embodiment of the Prosthesis Extraction Apparatus. More particularly, FIG. 39 depicts the assembly of the fifth embodiment Prosthesis Extraction Apparatus 10E with a surgical hammer 180 in position over the rotatable knurled handle 182 connected by an extension shaft 184 that drives the miter gear system 186. The miter gear box 188 is the means to move the knife blades 12A in and out together using left hand threaded drive shaft 190 and right hand threaded drive shaft 192. The three miter gear heads 194 are connected to the left hand threaded drive shaft 190, the right hand threaded drive shaft 192 and the extension shaft 184 from the rotatable handle 182. The knife blades 12A are held by the threaded knife blocks 196. The knife blades 12A are able to move in the miter gear box 188 by the means of the openings on the top 198 and openings on the bottom surface 200.

FIG. 40 depicts an enlarged partial cross-sectional view of the fifth embodiment of the Prosthesis Extraction Apparatus. As per FIG. 39, here in FIG. 40 there is shown more close-up detail of the rotatable knurled handle 182 connected by an extension shaft 184 that drives the Miter gear system 186. The miter gear box 188 is the means to move the knife blades 12A in and out together using left hand threaded drive shaft 190 and right hand threaded drive shaft 192. The three miter gear heads 194 are connected to the left hand threaded drive shaft 190, the right hand threaded drive shaft 192 and the extension shaft 184 from the rotatable handle 182. The knife blades 12A are held by the threaded knife blocks 196. The knife blades 12A are able to move in the miter gear box 188 by the means of the openings on the top 198 and openings on the bottom surface 200.

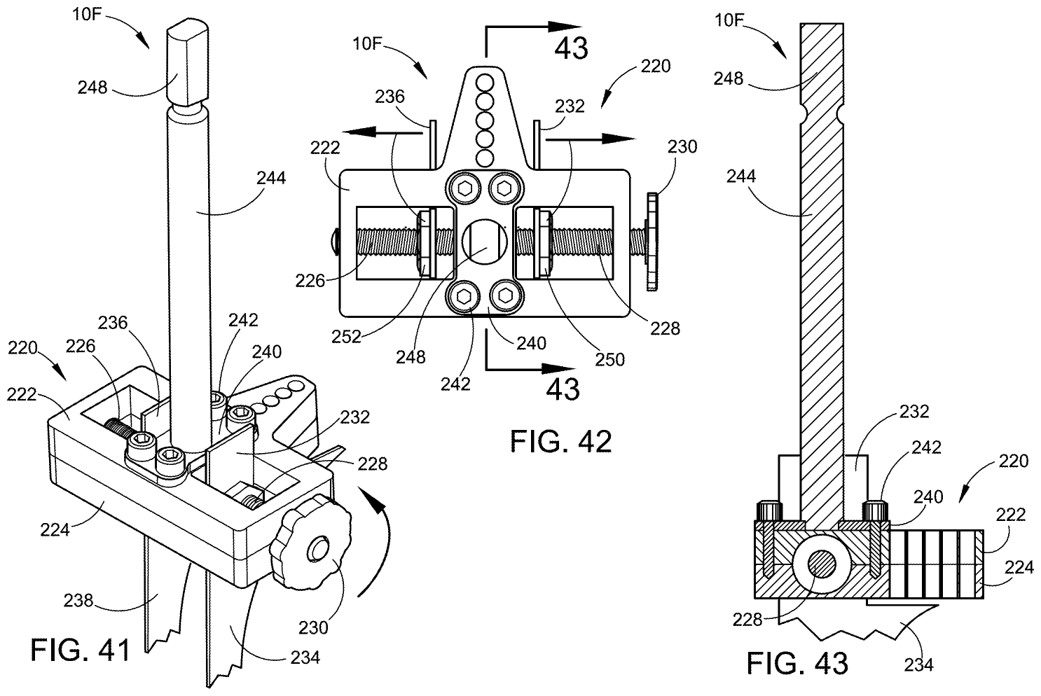

FIG. 41 depicts a partial top and front perspective view of a sixth embodiment of the Prosthesis Extraction Apparatus 10F surgical knife blade mounting and support block having a two-piece adjustable H-block 220 housing a rotatable threaded rod having right hand threads 226 on one end and left hand threads 228 on the opposite end, and a single adjustment knob 230 on one side. The two-piece H-block 220 is constructed by sandwiching the threaded rod having right hand threads 226 and left hand threads 228 between an upper housing portion 222 and a lower housing portion 224. The upper housing portion 222 and lower housing portion 224 are held together by Allen screws 242 threaded through an assembly plate 240 and into the upper housing 222 and lower housing 224 pieces, making the H-block 220 a two-piece assembly. Prior to assembly, each of the two surgical knife blades 234 and 238 are threaded onto each of the threaded rod left and right handed threads 228 and 226, respectively. The upper sections of each surgical knife blade 232 and 236 extend upward out of and above the upper housing portion 222 and are visualized by the user to determine position of the two surgical knife blades when adjustments are made. Adjustments are made by rotating the knob 230 causing the threaded nut attached (see FIG. 43) surgical knife blades 234 and 238 to move outwardly and inwardly together in unison (see FIG. 42). Osteotome mounting shaft 244 is affixed to assembly plate 240 and has an osteotome attachment point 248.

FIG. 42 depicts a top view of a sixth embodiment of the Prosthesis Extraction Apparatus shown 10F surgical knife blade mounting and support block shown in FIG. 41, having a two-piece adjustable H-block 220 with a rotatably embedded threaded rod having right hand threads 226 and left hand threads 228 located between an upper housing portion 222 and a lower housing portion 224 (not shown). The upper section of each surgical knife blade 232 and 236 includes a welded on threaded nut 250 and 252, respectively. A left hand half and right hand half threaded rod 228 and 226 have opposing threads (right and left handed threads on opposite ends) and when knob 230 is rotated, surgical knife blades 232 and 236 move simultaneously and proportionally outward or inward (see motion indication arrows) along threaded rods 226 and 228 depending on the rotation of knob 230 clockwise or counter clockwise. The upper and lower housing portions are assembled by Allen screws 242 extending through an assembly plate 240 and into both the upper and lower housing portions. At the top of osteotome mounting shaft 244 (not seen here, see FIG. 43) is an osteotome attachment point 248.

FIG. 43 depicts a partial cross-sectional view of a sixth embodiment of the Prosthesis Extraction Apparatus 10F surgical knife blade mounting and support block shown in FIG. 42, having a two-piece adjustable H-block 220 having an upper housing portion 222 and a lower housing portion 224 when assembled as shown rotatably sandwiching a left hand half and right hand half threaded rod 228 therein. Upper housing portion 222 and a lower housing portion 224 are held together using Allen screws 242 which extend down through an assembly plate 240 and into both the upper housing portion 222 and a lower housing portion 224. The configuration of the surgical knife blades is shown in greater detail in FIG. 45 through FIG. 48.

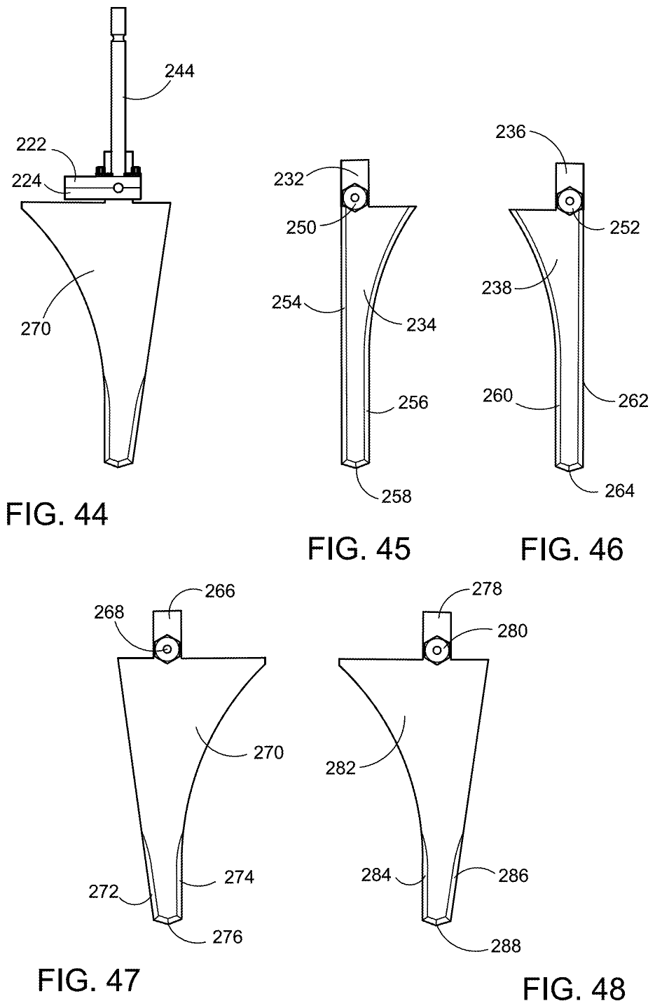

FIG. 44 depicts a side elevational view of a sixth embodiment of the Prosthesis Extraction Apparatus 10F surgical knife blade mounting and support block, having a two-piece adjustable H-block 220 with a rotatably embedded threaded rod having right hand threads 226 and left hand threads 228 located sandwiched between an upper housing portion 222 and a lower housing portion 224. The adjustment knob (not shown here) is on the other side of the H-block housing. Surgical knife blade 270 is attached as described in FIGS. 41 and 42 above. The entire assembled adjustable H-block and surgical knife blade 270 is attachable to an osteotome power tool (electric or pneumatic) on shaft 244.

FIG. 45 depicts a surgical knife blade 234 with a threaded nut 250 for attachment to the sixth embodiment of the Prosthesis Extraction Apparatus 10F surgical knife blade mounting and support block having a two-piece adjustable H-block with a threaded rod and single adjustment knob (not shown). This surgical knife blade 234 has an upper section 232 and is sharpened on the front side 254 and the backside 256. The end of the surgical knife blade has a beveled sharpened tip 258.

FIG. 46 depicts a surgical knife blade 238 which would be used opposite to the knife blade shown in FIG. 45, having a threaded nut 252 for attachment to the sixth embodiment of the Prosthesis Extraction Apparatus 10F having a two-piece adjustable H-block with a threaded rod and single adjustment knob (not shown). This surgical knife blade 238 has an upper section 236 and is sharpened on the front side 260 and the backside 262. The cutting end of the surgical knife blade has a beveled sharpened tip 264.

FIG. 47 depicts an alternate larger surgical knife blade 270 having an upper section 266 wherein a threaded nut 268 has been affixed. This knife blade 270 is also attachable to the sixth embodiment of the Prosthesis Extraction Apparatus 10F surgical knife blade mounting and support block having a two-piece adjustable H-block with an embedded sandwiched threaded rod and single adjustment knob (not shown). This surgical knife blade 270 has an upper section 266 and is partially sharpened on the lower front side 272 and partially sharpened on the lower backside 274. The cutting end of the surgical knife blade has a beveled sharpened tip 276.

FIG. 48 depicts an alternate larger surgical knife blade 282 which would be used opposite to the knife blade 270 shown in FIG. 47, also having an upper section 278 wherein a threaded nut 280 has been affixed. This surgical knife blade 282 is also attachable to the sixth embodiment of the Prosthesis Extraction Apparatus 10F surgical knife blade mounting and support block having a two-piece adjustable H-block with an embedded sandwiched threaded rod and single adjustment knob (not shown). This surgical knife blade 282 has an upper section 278 and is partially sharpened on the lower front side 284 and partially sharpened on the lower backside 286. The cutting end of the surgical knife blade has a beveled sharpened tip 288.

The Prosthesis Extraction Apparatus 10A through 10F shown in the drawings and described in detail herein disclose arrangements of elements of particular construction and configuration for illustrating preferred embodiments of structure and method of operation of the present application. It is to be understood, however, that elements of different construction and configuration and other arrangements thereof, other than those illustrated and described may be employed for providing the Prosthesis Extraction Apparatus 10A through 10E in accordance with the spirit of this disclosure, and such changes, alternations and modifications as would occur to those skilled in the art are considered to be within the scope of this design as broadly defined in the appended claims.

While certain embodiments of the Prosthesis Extraction Apparatus have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. For example, one portion of one of the embodiments described herein can be substituted for another portion in another embodiment described herein. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Further, the purpose of the foregoing abstract is to enable the U.S. Patent and Trademark Office, foreign patent offices worldwide and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

I claim:

1. A prosthesis extraction apparatus, comprising:
(a) a mounting rod affixed to a blade accepting support block member; and
(b) one or more surgical knife blades removably attached to said support block member.

2. The prosthesis extraction apparatus according to claim 1, wherein said blade accepting support block member further includes an adjustment rod rotatably secured to said blade accepting support block member, and further wherein said two surgical knife blades are moveably attached to said rod rotatably secured to said blade accepting support block thereby enabling surgical knife blade width adjustment movement.

3. The prosthesis extraction apparatus according to claim 2, wherein said adjustment rod is a threaded rod.

4. The prosthesis extraction apparatus according to claim 3, wherein said threaded rod includes two sections, a first section and a second section, wherein said first section is configured having right-handed threads and said second section is located opposite said first section and is configured having left handed threads.

5. The prosthesis extraction apparatus according to claim 4, wherein said threaded rod is attached to one or more rotatable adjustment knobs, such that rotation of said one or more adjustment knobs causes rotation of said threaded rod.

6. The prosthesis extraction apparatus according to claim 5, wherein said surgical knife blades include a threaded portion and said two surgical knife blades are thereby threaded on to said threaded rod enabling said two surgical knife blades to be moveably attached to said threaded rod such that the width between said two surgical knife blades can be adjusted.

7. The prosthesis extraction apparatus according to claim 4, further including a miter gearbox housing having three right angle miter gears, a first miter gear, a second miter gear and a third miter gear, with the first miter gear attached to said mounting rod, the second miter gear attached to said first section of said threaded rod having right-handed threads and the third miter gear attached to said second section having left handed threads, such that when said mounting rod is manually rotated the first miter gear attached thereto actuates the second and third miter gears attached to said first and second sections of said threaded rod, thereby enabling width adjustment of the surgical blades attached to said threaded rods.

8. The prosthesis extraction apparatus according to claim 1, wherein said mounting rod is affixable to a surgical osteotome power tool for surgical osteotome power tool assisted prosthesis extraction operations.

9. The prosthesis extraction apparatus according to claim 7, wherein said mounting rod is affixable to a handle for manual surgical knife blade adjustment and hand-held prosthesis extraction operations.

10. The prosthesis extraction apparatus according to claim 1, wherein said blade accepting support block further includes an alignment bar.

11. A prosthesis extraction apparatus, comprising:
(a) surgical knife blade mounting and support block having a two-piece adjustable H-block housing a rotatable threaded rod having right hand threads on one end and left hand threads on the opposite end, and a single rotatable adjustment knob connected to said rotatable threaded rod; and
(b) two surgical knife blades having an upper blade portion and a lower blade portion wherein said upper blade portion includes a threaded section and said lower blade portion includes a sharpened cutting edge;
wherein said two surgical knife blades are attached to each of said threaded rod end by the upper blade portion threaded section and when said adjustment knob is rotated this causes the threaded rod to rotate and this causes the surgical knife blades to move inwardly decreasing the distance between said two surgical knife blades and outwardly increasing the distance between said two surgical knife blades, thereby enabling adjustment of the distance between said two surgical knife blades.

12. A method for making a prosthesis extraction apparatus, comprising the steps of:
(a) providing a mounting rod affixed to a blade accepting support block member; and
(b) providing one or more surgical knife blades removably attached to said support block member.

13. The method for making a prosthesis extraction apparatus according to claim 12, wherein said blade accepting support block member further includes an adjustment rod rotatably secured to said blade accepting support block member, and further wherein said two surgical knife blades are moveably attached to said rod rotatably secured to said blade accepting support block thereby enabling surgical knife blade width adjustment movement.

14. The method for making a prosthesis extraction apparatus according to claim 13, wherein said adjustment rod is a threaded rod.

15. The method for making a prosthesis extraction apparatus according to claim 14, wherein said threaded rod includes two sections, a first section and a second section, wherein said first section is configured having right-handed threads and said second section is located opposite said first section and is configured having left handed threads.

16. The method for making a prosthesis extraction apparatus according to claim 15, wherein said threaded rod is attached to one or more rotatable adjustment knobs, such that rotation of said one or more adjustment knobs causes rotation of said threaded rod.

17. The method for making a prosthesis extraction apparatus according to claim 16, wherein said surgical knife blades include a threaded portion and said two surgical knife blades are thereby threaded on to said threaded rod enabling said two surgical knife blades to be moveably attached to said threaded rod such that the width between said two surgical knife blades can be adjusted.

18. The method for making a prosthesis extraction apparatus according to claim 15, further including a miter gearbox housing having three right angle miter gears, a first miter gear, a second miter gear and a third miter gear, with the first miter gear attached to said mounting rod, the second miter gear attached to said first section of said threaded rod having right-handed threads and the third miter gear attached to said second section having left handed threads, such that when said mounting rod is manually rotated the first miter gear attached thereto actuates the second and third miter gears attached to said first and second sections of said threaded rod, thereby enabling width adjustment of the surgical blades attached to said threaded rods.

19. The method for making a prosthesis extraction apparatus according to claim 12, wherein said mounting rod is affixable to a surgical osteotome power tool for surgical osteotome power tool assisted prosthesis extraction operations.

20. The method for making a prosthesis extraction apparatus according to claim 18, wherein said mounting rod is affixable to a handle for manual surgical knife blade adjustment and hand-held prosthesis extraction operations.
